(19) United States
(12) Patent Application Publication
(10) Pub. No.: US 2026/0367377 A1
(43) Pub. Date: Nov. 19, 2026

(54) METHOD AND SYSTEM FOR THE OPTICAL CHARACTERIZATION OF THIN SAMPLES

(71) Applicants: YPF TECNOLOGÍA S.A., Ciudad Autónoma de Buenos Aires (AR); UNIVERSIDAD DE BUENOS AIRES, Ciudad Autónoma de Buenos Aires (AR); CONSEJO NACIONAL DE INVESTIGACIONES CIENTÍFICAS Y TÉCNICAS (CONICET), Ciudad Autónoma de Buenos Aires (AR)

(72) Inventors: Esteban Alejo DOMENÉ, Berisso Pcia. de Buenos Aires (AR); Hernan Edgardo GRECCO, Ciudad de Buenos Aires (AR); Juan Marco BUJJAMER, Ciudad de Buenos Aires (AR); Joaquin TORRES, Ciudad de Buenos Aires (AR); Alejandro MAZZEO, Ciudad de Buenos Aires (AR); Ezequiel GONZÁLEZ PELEGRI, Berisso Pcia. de Buenos Aires (AR); Carlos Gabriel GRASETTI PALACIO, Berisso Pcia. de Buenos Aires (AR)

(21) Appl. No.: 19/208,160

(22) Filed: May 14, 2025

(30) Foreign Application Priority Data
Apr. 18, 2024 (AR) P20240100983

(51) Int. Cl. G02B 21/00; G01N 33/24; G02B 21/16; G02B 21/36

(52) U.S. Cl. CPC G02B 21/0072; G02B 21/0032; G02B 21/0068; G02B 21/16; G02B 21/367; G01N 33/24

(57) ABSTRACT

The present description discloses a microscopical method which applies Fourier ptychography for analyzing thin samples such as those associated to digital petrography and digital pathology. The description additionally provides an integrated system able to carry out the aforementioned method.

### FIELD OF THE INVENTION

The present invention generally relates to the field of digital microscopy, particularly to the field of the characterization of thin samples by means of digital microscopy. More specifically, the invention refers to a microscopical method which applies Fourier ptychography for analyzing thin samples such as those associated to digital petrography and digital pathology.

### BACKGROUND OF THE INVENTION

In the field of the analysis of thin sections by microscopy techniques, the existing methods to obtain a large visual field with a high resolution exhibit several limitations for their proper implementation. In fact, the only existing manner in the state of the art to achieve this is by preparing a mosaic of images, and then combining them. This process typically takes a long time, exhibits frontier problems, and requires equipment with several mobile parts, such as a motorized plate. It is thus desirable to develop a technique which overcomes these shortcomings, by providing a way to obtain a single wide-field image with a high resolution, using relatively simple equipment and with an efficient processing of the images so as to optimize the time required to obtain a final image.

Particularly, the petrographic study of thin slices is a routine assay performed on any rock analysis. It allows to understand the prevalent lithology, induce transport features, classify the porosity by type and size, and characterize the poral textures, thus providing a visual microscopic inspection of the rock. The main technical problem exhibited by this technique is that the results tend to be subjective, and the quantification is performed on small areas which may not always be representative of the whole rock. Correspondingly, a technique with the advantages proposed above would be of great use for digital petrography. Similar considerations apply for other application wherein the provision of high-resolution wide-field images would be beneficial, such as digital pathology (i.e., the analysis of biomedical samples by digital microscopy).

Fourier ptychography is an image processing technique for optical microscopy which essentially consists in acquiring several wide-field, low-resolution images of a sample with different coherent illumination angles, and then processing the acquired images to obtain a high-resolution wide-field reconstructed image of the sample. Patent application WO 2014070656 A1 describe devices and methods for Fourier ptychography imaging. Patent EP 3146501 B1 describes a method for Fourier ptychographic microscopy, focusing mainly on the illumination method, which is performed by a light emitting diode array.

U.S. Pat. No. 11,010,883 B2 discloses a method for the automated analysis of petrographic thin section images based on a machine learning processing method for performing a segmentation of thin rock samples, but without any disclosure of a particular image acquisition method.

The reference “3D differential phase-contrast microscopy with computational illumination using an LED array”, Optics Letter; Vol. 39, No 5, 28 Feb. 2014, page 1326, DOI: 10.1364/OL.39.001326, by Lei Tian et al. discloses 3D differential phase-contrast (DPC) microscopy, based on computational illumination with a programmable LED array. By capturing intensity images with various illumination angles generated by sequentially patterning an LED array source, images may be digitally refocused through various depths via light field processing. The intensity differences from images taken at complementary illumination angles are then used to generate DPC images, which are related to the gradient of phase. The disclosed method achieves 3D DPC with simple, inexpensive optics and no moving parts.

The reference “Quantitative differential phase contrast imaging in an LED array microscope”, Optics Express, Vol. 23, No 9, 4 May 2015, DOI: 10.1364/OE.23.011394, by Lei Tian et al. discloses a 2D DPC method using asymmetric illumination patterns. The document discloses the derivation of the 2D weak object transfer function (WOTF) and the development of a quantitative phase reconstruction method that is robust to noise. The effect of spatial coherence is studied experimentally, and multiple-angle DPC is shown to provide improved frequency coverage for more stable phase recovery. The disclosed method uses an LED array microscope to achieve real-time (10 Hz) quantitative phase imaging with in vitro live cell samples.

### SUMMARY OF THE INVENTION

The present invention provides a method for the optical characterization of a thin sample, the method comprising:
i- providing an optical transmission microscope comprising a low magnification objective, and an illuminator comprising an array of LEDs which may be turned on in a controlled manner to provide different illumination patterns;
ii- acquiring a plurality of low-resolution images of the thin sample by means of the optical transmission microscope, wherein each image is taken with a different illumination pattern;
iii- processing the plurality of low-resolution images to obtain a reconstructed high-resolution image of the thin sample, wherein the reconstructed high-resolution image contains both amplitude and phase information of the sample; and
iv- analyzing the reconstructed high-resolution image of the thin sample according to one or more quantification pipelines.

In an embodiment of the invention, the thin sample is selected from a thin section of a rock and a thin section of a biomedical sample. Preferably, the thin sample is a thin section of a rock.

In an embodiment of the invention, the illuminator comprises multispectral LEDs which may be turned on sequentially.

In an embodiment of the invention, the one or more quantification pipelines comprise porosity determination, pore and pore throat discrimination, segmentation in grains, mineralogic determination of components and/or identification of regions of interest.

In another embodiment of the invention, the method further comprises a calibration step prior to processing the plurality of low-resolution images according to step iii, wherein the calibration step comprises the sub-steps of:
a) simulating a group of images from a model of a standard sample, introducing a deliberate decalibration by means of decalibrated parameters;
b) reconstructing an image of the standard sample without using the decalibration;
c) comparing the model of the standard sample with the reconstructed image to classify the differences according to the introduced decalibration; and
d) obtaining a combined estimator from the decalibrated parameters, wherein the estimator is then used to estimate a decalibration source in the reconstructed high-resolution image of the thin sample.

It is another aspect of the invention to provide a system for the optical characterization of thin samples, the system comprising:
(a) an optical transmission microscope comprising a low magnification objective, and an illuminator comprising an array of LEDs which may be turned on in a controlled manner to provide different illumination patterns;
(b) a camera configured to acquire images from the optical transmission microscope; and
(c) a processing unit with data connection to both the illuminator and the camera;
   wherein the processing unit is configured to carry out the following steps:
i- controlling the illuminator to provide different illumination patterns;
ii- acquiring a plurality of low-resolution images of the thin sample by means of the optical transmission microscope, wherein each image is taken with a different illumination pattern;
iii- processing the plurality of low-resolution images to obtain a reconstructed high-resolution image of the thin sample; and
iv- analyzing the reconstructed high-resolution image of the thin sample according to one or more quantification pipelines.

In an embodiment of the invention, the illuminator comprises multispectral LEDs which may be turned on sequentially. Preferably, the multispectral LEDs are disposed on the illuminator in a spherical arrangement in which the sample is at the center. More preferably, the multispectral LEDs comprise RGB LEDs.

In an embodiment of the invention, the optical transmission microscope further comprises a polarizing module, wherein the polarizing module comprises two polarizers which can be rotated independently.

### BRIEF DESCRIPTION OF THE DRAWINGS

FIG. 1. Diagram describing end-to-end high image capture to final output.

FIG. 2. Gold Nano Lithography sample. Top Left—Original image from conventional optical microscope; Top Right—Magnitude and phase image reconstruction from Fourier ptychography. Bottom—Height profiles of image inset (dark circle in larger image): a comparison between the original image, the Fourier ptychography image (FPM) and an Atomic Force Microscope (AFM).

FIG. 3. Liquid medium drop of C. Elegans sample in microscope slide with cover glass (not in vivo). TetraSpeck Fluorescent Microspheres 505/515 nm (green) 4.0 μm (T7283 ) on microscope slide with cover glass.

FIG. 4. Full specimen reconstruction of C. Elegans sample. The inset contains a digitally magnified image of the section within the box.

### DETAILED DESCRIPTION OF THE INVENTION

The present disclosure describes a method for the optical characterization of thin samples based on Fourier ptychography (FP).

As described above, FP essentially consists in acquiring several wide-field, low-resolution images of a sample with different coherent illumination angles, and then processing the acquired images to obtain a high-resolution wide-field reconstructed image of the sample. To obtain the required different illumination angles, the method of the invention contemplates using a fixed illuminator with an array of single light sources which may be turned on and off either independently or in small groups at a time to generate different illumination patterns. The selected illumination angles are not completely arbitrary but shall satisfy an information overlapping criterion determined by mainly two parameters: the distance between the illuminator and the sample and the numerical aperture of the objective used.

Correspondingly, the method according to the invention comprises a first step of providing an optical transmission microscope comprising a low magnification objective, and an illuminator comprising an array of LEDs which may be turned on in a controlled manner to provide different illumination patterns.

The low magnification objective is essential to obtain a wide-field image of the sample, which will be of low resolution due to the limitations of such magnification objectives known to those of skill in the art. Preferably, the magnification of the low magnification objective is 1× or 2× with the aim of maximizing the captured area of the image. A key characteristic of this technique is given by the need of a low numerical aperture objective (0.1, 0.075, or below), this desensitizes the focusing of the sample but limits the geometry of the illuminator. Concretely, the quotient between the numerical aperture and the wavelength (NA/λ) of the incoming light defines the diameter of the pupil in the reciprocal (Fourier) space, while the displacement of this pupil is proportional to the sine of the angle of the light source. With these parameters, an overlap higher than 70% between subsequent images is required for the algorithms to converge.

The illuminator of the microscope is an array of LEDs wherein each LED may be turned on independently and sequentially, or in small groups at a time. The array should have a shape and arrangement so as to ensure an even illumination from any LED. As long as the aforementioned proviso is fulfilled along with the overlapping requirement, the LEDs may be positioned in any arrangement within the array. In a preferred embodiment, the LEDs are disposed in a spherical arrangement in which the sample is at the center. Spacing of the LEDs in the sphere is chosen in order to ensure homogeneous full coverage of the spatial frequency space, thus ensuring at least 60% area overlap between neighboring LEDs. When faster imaging is required, a fraction of the LEDs can be used but with a lesser image quality in low signal to noise ratio samples.

Depending on the nature of the samples to image, it may be convenient for the LEDs of the illuminator to be able to provide light with different wavelengths, which is useful for identifying components of the sample. Typical histopathology samples can be studied in the visible range and thus RGB LEDs are the right choice for them. Petrological thin sections need a broader range in order to distinguish its composing elements in which case the illuminators should be multispectral (visible including ultraviolet and near-infrared bands). Correspondingly, in a preferred embodiment of the invention, the LEDs of the illuminator are multispectral LEDs. More preferably, the multispectral LEDs comprise RGB LEDs, so that the resulting digital image is comparable with the one obtained by standard microscopy. The term “RGB LEDs”, may refer to combined LEDs consisting of a red, green and blue LEDs, or to individual red, green and blue LEDs. The use of individual red, green and blue LEDs allows the use of high optical power LEDs, which may be rotated around the sample to change the incidence angle thereof, thus ensuring a complete sampling. In a particularly preferred embodiment of the invention, the multispectral LEDs additionally comprise other wavelengths in the visible spectrum as well as UV And IR to increase the contrast between materials that appear similar in RGB.

The method comprises a second step of acquiring a plurality of low-resolution images of the thin sample by means of the optical transmission microscope, wherein each image is taken with a different illumination pattern, thus imposing the so called “phase diversity” in the capturing process. Each acquisition is a low-resolution image captured by an individual (or a small group of) LED at a particular wavelength in a predefined sequence, preferably spiraling out from the center of the illuminator array.

The acquisition is performed by means of a detector, which typically is a camera such as a CMOS or CCD camera. A person of skill in the art will appreciate that the detector must comply with certain requirements in order to be used in a method based on FP such as the one described herein. Specifically, the detector should comply with three main characteristics:
1. The required pixel size should satisfy the Nyquist-Shannon criterion. Briefly, said criterion establishes that the spatial sampling resolution should be at least half the minimum detail observable by the system, and this is proportional to the wavelength and inversely proportional to twice the numerical aperture. The pixel size should be selected for the most demanding of the wavelengths utilized in the application (the shortest one).
2. Additionally, considering that FP uses objectives with low numerical aperture and large field of view, it is essential for the sensor to be large enough to capture the whole field of view given the selected magnification. Small magnification objectives are preferred whenever there is a correspondently large sensor to acquire the full sampling area.
3. The spectral range of the detector should match that of the illuminator selected for the application. The most demanding case is that of ultraviolet or near-infrared bands where special detectors should be considered.

As the objective of the optical transmission microscope provided for the method is of low magnification and low numerical aperture, each individual image acquired will have a low resolution. The method requires the acquisition of a plurality of such low-resolution images for later processing to obtain a higher resolution reconstructed image. Such variability of images should account for the phase diversity of the imaged sample, where each capture contains not only a low-resolution version of it but unique view of the detail of the sample. This is achieved by the method of the invention by acquiring each low-resolution image with a different illumination pattern, as explained above.

The number of low-resolution images acquired will have an impact on the resolution of the reconstructed image, with a larger number of low-resolution images providing a reconstructed image with higher resolution, at the expense of a longer time necessary for obtaining the high-resolution reconstructed image. The number of low-resolution images acquired depends on the type of sample and desired resolution for the reconstructed high-resolution image. Typically, the number of low-resolution images acquired may vary within the range of 1 to about 100 for each color of the LED array of the illuminator. In the embodiment wherein the LEDs of the array are independent RGB LEDs, the number of low-resolution images may thus typically vary between 3 to about 300.

Each image must be acquired with a different illumination pattern. As each individual LED of the array of the illuminator may be turned on and off independently, according to the invention an “illumination pattern” should be understood as a particular distribution of lit and unlit LEDs on the array, generating a particular distribution of light intensity reaching the sample and the detector. When the LEDs are multispectral LEDs, the term “illumination pattern” also encompasses a particular distribution of wavelengths in the light reaching the sample and the detector. This way, each low-resolution image acquired includes different information from the sample which is then used in the reconstruction of a high-resolution image. The illumination pattern used for each low-resolution image may be randomized, or it may be pre-defined according to certain operation parameters, as long as different incidence angles are scanned and the whole sample is illuminated. The information of which LEDs were lit for each illumination pattern is later used for the reconstruction step, so it is important for such information to be known and stored. A person of skill in the art is able to select a software among those existing in the state of the art suitable for such generation of illumination patterns.

After the plurality of low-resolution images have been acquired, the method of the invention comprises a third step of processing the plurality of low-resolution images to obtain a reconstructed high-resolution image of the thin sample. The reconstruction of the high-resolution image may be performed by any algorithm known in the art for such image processing. For example, the reconstruction may be carried out by means of a Gerchberg-Saxton (GS) algorithm, an Error Reduction (ER) algorithm, or an Extended Ptychographical Iterative Engine (ePIE) algorithm. Preferably, the reconstruction is carried out by means of an ePIE algorithm.

In order to achieve a proper reconstruction of the high-resolution image, it is essential for the parameters of the microscope to be known and fixed, since said parameters are used in the reconstruction algorithms. Any drift from their theoretical value yields an artifact in the final reconstruction, which may be difficult to isolate and correct a posteriori. Therefore, a proper calibration of the microscope is often necessary for properly carrying out the method of the invention. Correspondingly, in an embodiment of the invention, the method further comprises a calibration step prior to processing the plurality of low-resolution images according to the third step, wherein the calibration step comprises the sub-steps of:
a) simulating a group of images from a model of a standard sample, introducing a deliberate decalibration;
b) reconstructing an image of the standard sample without using the decalibration;
c) comparing the model of the standard sample with the reconstructed image to classify the differences according to the introduced decalibration; and
d) obtaining a combined estimator from the decalibrated parameters, wherein the estimator is then used to estimate a decalibration source in the reconstructed high-resolution image of the thin sample.

The model of a standard sample for proceeding with the calibration refers to either a sample of a known material which is used as “ground truth” for the optimization, or to any given sample which has soft, unstructured refraction index spatial variations.

The decalibration introduced for obtaining the images of the model of the standard sample may be associated with any of the parameters relevant to the application of the selected reconstruction algorithm. For instance, the decalibration may be a shift in the LEDs array (either by modifying the distance or the inclination thereof with respect to the sample), an inclination of the plate containing the sample, a modification in the relative distance between the sample and the objectives, a modification in the distance between the optical elements of the microscope, a modification in the effective numerical aperture and focal distance of said optical elements, among others.

The estimator obtained by the calibration step is the image of the sample itself, exhibiting particular patterns (such as lines, circles, curves, etc.), each of which is associated to a specific decalibration as those described in the previous paragraph.

The high-resolution image obtained by means of the method of the invention has a wide field of view, without the need for combining a mosaic of narrower-field-of-view images obtained for different sections of the samples, thus avoiding frontier issues which arise from imperfect overlapping of the different images combined. Additionally, the high-resolution image is multidimensional, also referred to as a spectrally resolved image, and it contains both amplitude and phase information of the sample. This can be seen as a deepening in the information contained in the sample as compared to regular microscopy, where on the one side there is spectrally resolved layers from where to analyze some aspects of the chemical composition of the sample, but also phase-resolved data containing information about its material structure and its thickness.

Obtaining such high-resolution wide-field reconstructed images is of particular use for thin samples such as those typically analyzed in digital petrography and digital pathology. To the inventors'knowledge, the use of a FP-based microscopic technique applied to petrographic thin samples has never been reported. Neither it has been disclosed the use of amplitude and phase information as obtained in such FP-based microscopic technique to characterize and then segment individual components.

The term thin sample refers to a sample that is thin enough to be able to observe using a transmission microscope. This will depend on the optical absorbance of the material and the power and spectrum of the illumination. For example, in general rock thin sections are sanded down until quartz grains appear white (this happens when the thickness is between 20 and 30 microns (Michel-Lévy, A., and Lacroix, A. (1888), Les Mineraux des Roches. Librairie Polytechnique Paris, 334 p). This enables the description of the thin samples to be compared to literature values and descriptions which are done for thickness values under 30 microns.

Correspondingly, the thin sample to be analyzed by the method of the invention is preferably selected from a thin section of a rock sample, or a thin section of a biomedical sample. More preferably, the thin sample is a thin section of a rock sample.

A thin section of a rock sample is a section obtained from a soil or rock sample of interest, which is typically in the order of tenths of microns thick, depending on the source and the available material. A thin section of a biomedical sample is a section of a pathological tissue of a subject, for instance a human subject, which is taken for its analysis for diagnostic purposes. Depending on the particular tissue sampled, and the pathology to be determined, a thin section of a biomedical sample may have different characteristics and widths. It is well within the knowledge of a person of skill in the arts of petrography and histopathology to obtain such thin samples.

Such a high-resolution image allows for a post-acquisition analysis thereof, which may be used to characterize the thin sample by means of several different features. Since the method of the invention is based on FP, the high-resolution image obtained comprises both amplitude and phase information of the sample. That is, for each pixel two observables are obtained which are directly linked to the complex refractive index. Particularly when measured at different wavelengths, the refractive index of a sample is a property which allows the identification of materials present therein. A physical model of the sample is obtained by assuming that each resoluble point of the microscope has a complex refractive index for each wavelength, and that the variation rate of the sample thickness is low throughout the whole sample. Then, a proxy of the refractive index is calculated in each point taking into account the ratio between the refractive index and the wave dephasing, thus allowing the estimation of the material in each point of the sample. A relative absorption for each wavelength can be calculated as the amplitude ratio between each pixel and the average of a group of reference pixels. Additionally, the relative index of refraction between wavelengths in each point can be calculated by dividing the corresponding phase values. This information may thus be used to identify and separate groups of pixels according to their morphological, colorimetric and textural properties by means of segmenting and clustering algorithms. These segmented images may then be analyzed to determine certain relevant features of the sample. The algorithms used for segmenting and clustering the images as described are known in the art, and may comprise, for instance, constructing a color- or roughness-based cluster for neighboring pixels, region growing, k-means clustering, mean-shift clustering, quick-shift, among others. Additionally, human-trained machine learning algorithms may be used. Examples of such algorithms may be found, for instance, in https://sbme-tutorials.github.io/2019/cv/notes/6_week6.html and https://scikit-image.org/docs/stable/api/skimage.segmentation.html. The process of segmenting, clustering and analyzing the segmented images for each feature of interest is referred to a “pipeline” or a “quantification pipeline”. Since many features may be determined from the post-acquisition analysis of the high-resolution image, the method of the invention comprises a fourth step of analyzing the reconstructed high-resolution image of the thin sample according to one or more quantification pipelines. The process of segmenting, clustering and analyzing the segmented images for each feature of interest is referred to a “pipeline” or a “quantification pipeline”. Since many features may be determined from the post-acquisition analysis of the high-resolution image, the method of the invention comprises a fourth step of analyzing the reconstructed high-resolution image of the thin sample according to one or more quantification pipelines.

A clear advantage of this system that contains the complete chain of the sample reconstruction to the final decomposition is the ability to incorporate the obtained segmented output in the initial reconstruction as a feedback method. In this way, known and pre-characterized samples can be used to improve the performance of the device in a semi-automatic fashion.

The features used to characterize the thin sample in the post-acquisition analysis depend on the type of sample. For instance, if the thin sample is a thin section of a rock sample, the features used to characterize the thin sample may comprise porosity, connectivity of the poral network, grain size, among others. If, alternatively, the thin sample is a thin section of a biomedical sample, the features used to characterize the thin sample typically comprise a colorimetric and morphological analysis of different dyed tissues and elements such as cells, tumors, etc.

Correspondingly, if the thin sample is a thin section of a rock sample the one or more quantification pipelines applied for analyzing the reconstructed high-resolution image may comprise porosity determination, segmentation in grains and/or identification of regions of interest.

The present invention additionally provides a system for the optical characterization of thin samples which is capable of carrying out the method described above. The system comprises not only the microscope with the illuminator as described above for the method of the invention, but also a camera for acquiring low-resolution images from the microscope, as well as a processing unit for controlling both the illuminator and the camera, reconstructing a high-resolution image from the low-resolution images acquired, and performing a post-acquisition analysis of the reconstructed high-resolution image. Correspondingly, the system of the invention comprises:
(a) an optical transmission microscope comprising a low magnification objective, and an illuminator comprising an array of LEDs which may be turned on in a controlled manner to provide different illumination patterns;
(b) a camera configured to acquire images from the optical transmission microscope; and
(c) a processing unit with data connection to both the illuminator and the camera;
wherein the processing unit is configured to carry out the following steps:
i- controlling the illuminator to provide different illumination patterns;
ii- acquiring a plurality of low-resolution images of the thin sample by means of the optical transmission microscope, wherein each image is taken with a different illumination pattern;
iii- processing the plurality of low-resolution images to obtain a reconstructed high-resolution image of the thin sample; and
iv- analyzing the reconstructed high-resolution image of the thin sample according to one or more quantification pipelines.

The same considerations described above for the optical microscope of the method of the invention apply as well to the optical microscope of the system. Correspondingly, in an embodiment of the invention, the illuminator comprises multispectral LEDs which may be turned on sequentially. Preferably, the multispectral LEDs are the LEDs are disposed in a spherical arrangement in which the sample is at the center. More preferably, the multispectral LEDs comprise RGB LEDs. As mentioned above, if the RGB LEDs are individual red, green and blue LEDs, this allows the use of high optical power LEDs. In a particularly preferred embodiment of the invention, the multispectral LEDs additionally comprise other wavelengths in the visible spectrum as well as UV And IR to increase the contrast between materials that appear similar in RGB. The system may additionally comprise a rotating curved arm to be able to change the incidence angle by turning on different LEDs and rotating the arm around 360° to reach every part of the sample.

It may be convenient to additionally include polarizers in the optical microscope of the system of the invention. Therefore, in another embodiment of the invention, the optical transmission microscope further comprises a polarizing module, wherein the polarizing module comprises two polarizers which can be rotated independently to discriminate between materials according to their optical activity. Such a design for the polarizing module allows to automatize the acquisitions at predefined angles and speed up the data acquisition.

As discussed above for the method of the invention, the camera of the system of this aspect of the invention may be selected from a CMOS or CCD camera.

The processing unit of the system is able to control both the illuminator and the camera of the system, so as to facilitate the acquisition of a plurality of low-resolution images of the sample, wherein each image is taken with a different illumination pattern. Therefore, the processing unit may contain instructions for generating a series a different illumination patterns in the illuminator, either according to a certain pre-defined program or randomly, while acquiring an image of the sample for each generated pattern, thus obtaining the plurality of low-resolution images.

The processing unit is also responsible for the processing of the plurality of low-resolution images according to the algorithms described above, so as to obtain a wide-field high-resolution image of the sample. To such end, the parameters of the microscope as obtained and optimized according to the calibration process described above may be entered to the processing unit as inputs for the processing of the images.

The processing unit is also able to carry out the post-acquisition analysis of the processed high-resolution image according to the pipelines described above. Correspondingly, the processing unit contains instructions for running the corresponding pipelines according to the segmenting and clustering algorithms relevant for the features of interest to be analyzed in the high-resolution image.

The processing unit may additionally be in data connection to a display device for the user to be able to input the necessary parameters both for processing the plurality of low-resolution images and for carrying out the post-acquisition analysis of the reconstructed high-resolution image, as well as visualize the plurality of low-resolution images acquired, the processed high-resolution image, and the results of the post-acquisition analysis, by means of an appropriate software.

The present invention exhibits several advantages when compared to other imaging techniques used in the art of digital petrography and digital pathology, such as:
it allows measuring both amplitude and phase of the sample;
it allows the 3D reconstruction of structures such as pores, due to the fact that information from different planes is collected;
an a posteriori focus on the image may be performed to correct any movement or blurring;
it is capable of refocusing on different layers of the sample and so capture axially resolved structures;
by measuring only a few angles, every polarization angle may be obtained a posteriori, thus allowing a post-acquisition inspection.

### End to End Description and Applications

As described, the microscopy system of the invention (as well as the related method) works as an end-to-end unit for both pathology and petrography applications. There are three main aspects shared by both types of samples that make them suitable for this common design, briefly:
1. The relative size of the minimum features. Both contain submicron features that are critical to resolve (cellular shapes, connections and coloration or cracks and incrustations in each case).
2. At the same time, the minimum features have to be resolved in the context of a large sample (1-inch or larger). The key features are frequently very dispersed and connected at very disparate scales.
3. There are traditional techniques that use visible (or nearly visible) spectrum to separate conditions or materials in the samples by their absorptivity at different wavelengths.
4. Thin samples (100 μm or less) can be obtained for both applications for the technique to work properly.

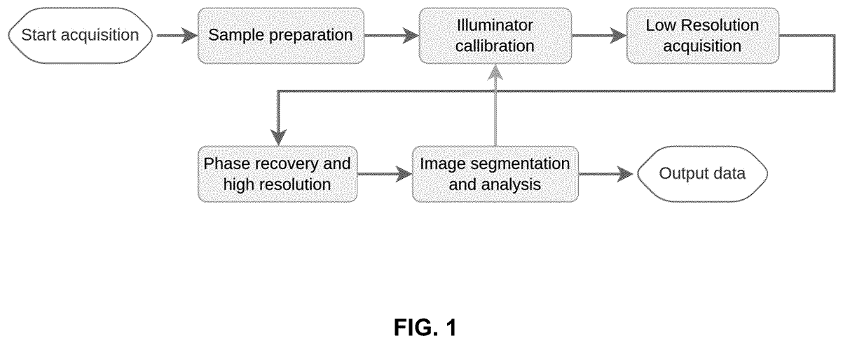

The method and system of the invention share a common workflow for both applications from the sample preparation to the final visualization/segmentation as shown in FIG. 1. It consists of:
a) Sample preparation. Thin samples of the tissue or rock sampled cut by specialized equipment.
b) Selection of the illumination parameters: wavelength, illumination geometry and pattern. The geometry is selected according to the selected objective and sensor and the illumination needed. The wavelengths to be used depend on the absorptive characteristics of the sample.
c) Calibration of the whole system with the given sample. This process can be manual (using a calibration pattern and expert knowledge) or automatic. The automatic variant is where one of the strengths of this end-to-end solution resides, as using the final reconstruction and segmentation of the initial acquisitions can be used to improve the imaging quality from the first stages of the capture process to the final ones.
d) Low resolution (and phase diversity) imaging. Physical capture of multiple images with different wavelengths and illumination angles. The time necessary to carry out this stage depends on the noise specifications of the sensor, the power of the individual LEDs and the transmission of the samples.
e) Phase recovery using the captured information. During this stage the phase recovery algorithm is applied over the collection of low-resolution samples to reconstruct the high-resolution image of the sample and its phase.
f) The final stage is the processing of the multiple layers of information of the capture to obtain the desired output information (segmentation, material properties and quantitative physicochemical characteristics of the sample).

### EXAMPLES

### Example 1—Reconstruction of a Gold Sample Deposited on a Substrate

The sample was placed on a plate and focused with a 1× objective of a transmission microscope equipped with a LED ring illuminator and a CCD camera. Once the sample was in focus, 200 images were taken with different illumination angles to fill the Fourier space, with at least 20% overlap between adjacent angles. For each illumination angle, the exposure time was set to use the camera's full dynamic range.

The reconstruction was performed using ePIE, with previously calibrated geometrical and optical parameters to obtain both magnitude and phase reconstructed images. The phase image does not contain relevant information given the opaque nature of the material. The sample was also analyzed with an atomic force microscope (AFM) for comparison.

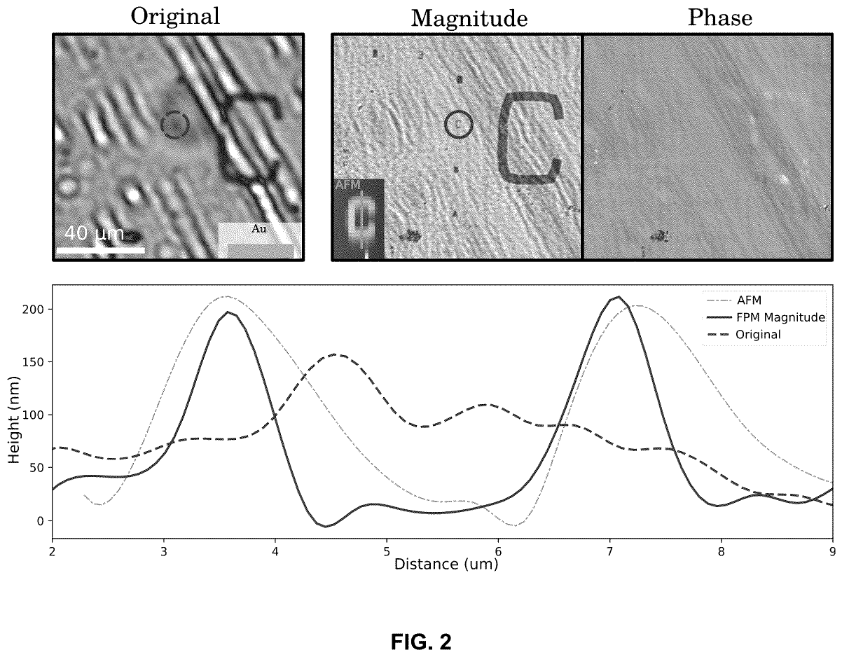

The obtained images and results are shown in FIG. 2.

### Example 2—Optimization of Images Acquired With a Ptychographic Microscope According to the Invention

For this experiment two different samples were used: a specimen of C. elegans and a sample consisting of a series of 3 μm beads on a surface. Each sample was placed on a plate and focused with a 1× objective of a transmission microscope equipped with a LED ring illuminator and a CCD camera. Once the sample was in focus, 200 images were taken with different illumination angles to fill the Fourier space, with at least 20% overlap between adjacent angles. For each illumination angle, the exposure time was set to use the camera's full dynamic range.

The reconstructions were performed using ePIE, with purposely inaccurate estimation of the geometrical and optical parameters to demonstrate the optimization process to recover the actual values.

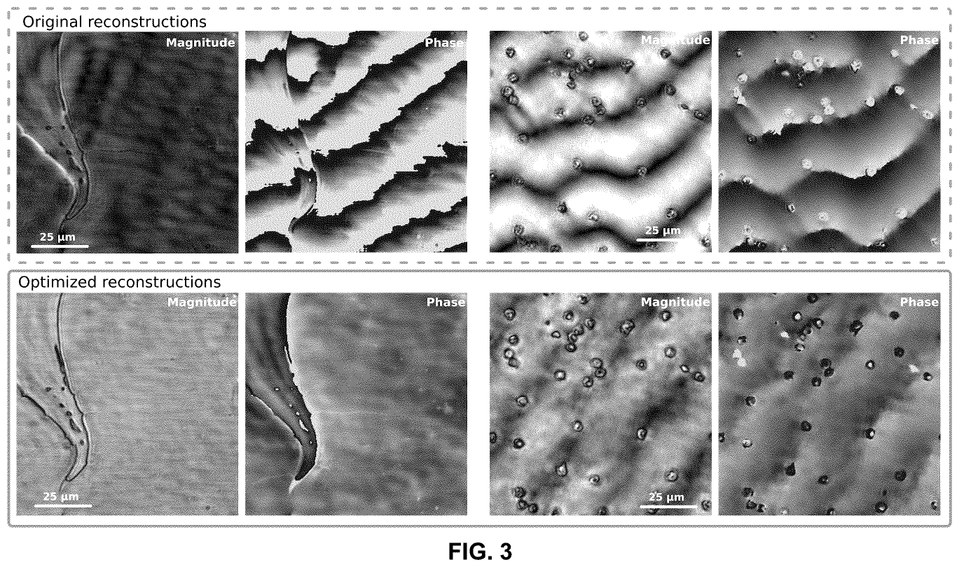

The obtained reconstructions are shown in FIG. 3. The images corresponding to the C. Eleganssample show a reduction in the diagonal bands in the phase reconstruction, as well as a contrast improvement in the magnitude reconstruction. For the reconstruction of the beads the reduction of interference fringes as a result of the optimization is evident.

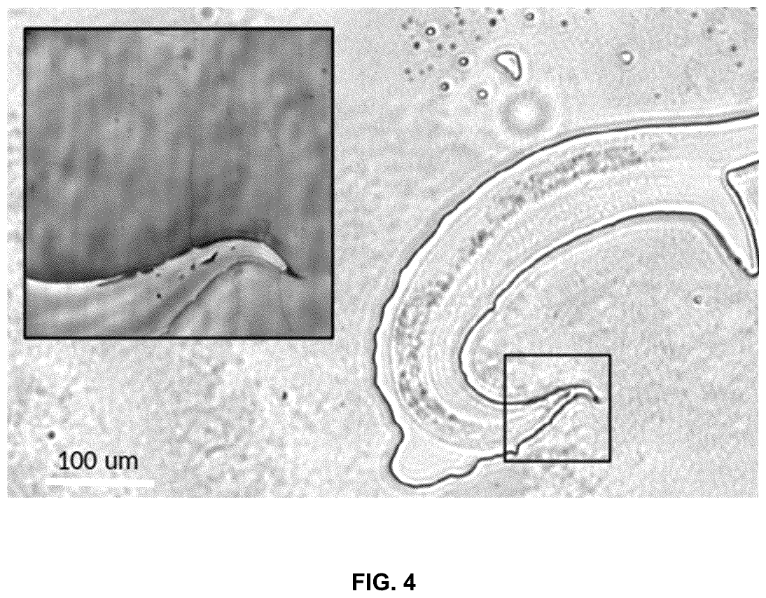

FIG. 4 shows the reconstructed image for the whole specimen of C. elegans, which proves the capacity for obtaining a high-resolution wide-field image by means of the present invention.

1. A method for the optical characterization of a thin sample, the method comprising:
i- providing an optical transmission microscope comprising a low magnification objective, and an illuminator comprising an array of LEDs which may be turned on in a controlled manner to provide different illumination patterns;
ii- acquiring a plurality of low-resolution images of the thin sample by means of the optical transmission microscope, wherein each image is taken with a different illumination pattern;
iii- processing the plurality of low-resolution images to obtain a reconstructed high-resolution image of the thin sample, wherein the reconstructed high-resolution image contains both amplitude and phase information of the sample; and
iv- analyzing the reconstructed high-resolution image of the thin sample according to one or more quantification pipelines.

2. The method of claim 1, wherein the thin sample is selected from a thin section of a rock sample and a thin section of a biomedical sample.

3. The method of claim 2, wherein the thin sample is a thin section of a rock sample.

4. The method of claim 1, wherein the illuminator comprises multispectral LEDs which may be turned on independently or in small groups.

5. The method of claim 1, wherein the one or more quantification pipelines comprise porosity determination, segmentation in grains, mineralogic determination of components and/or identification of regions of interest.

6. The method of claim 1, further comprising a calibration step prior to processing the plurality of low-resolution images according to step iii, wherein the calibration comprises the sub-steps of:
a) simulating a group of images from a model of a standard sample, introducing a deliberate decalibration;
b) reconstructing an image of the standard sample without using the decalibration;
c) comparing the model of the standard sample with the reconstructed image to classify the differences according to the introduced decalibration; and
d) obtaining a combined estimator from the decalibrated parameters, wherein the estimator is then used to estimate a decalibration source in the reconstructed high-resolution image of the thin sample.

7. A system for the optical characterization of thin samples, the system comprising:
(a) an optical transmission microscope comprising a low magnification objective, and an illuminator comprising an array of LEDs which may be turned on in a controlled manner to provide different illumination patterns;
(b) a camera configured to acquire images from the optical transmission microscope; and
(c) a processing unit with data connection to both the illuminator and the camera;
wherein the processing unit is configured to carry out the following steps:
i- controlling the illuminator to provide different illumination patterns;
ii- acquiring a plurality of low-resolution images of the thin sample by means of the optical transmission microscope, wherein each image is taken with a different illumination pattern;
iii- processing the plurality of low-resolution images to obtain a reconstructed high-resolution image of the thin sample; and
iv- analyzing the reconstructed high-resolution image of the thin sample according to one or more quantification pipelines.

8. The system of claim 7, wherein the illuminator comprises multispectral LEDs which may be turned on independently or in small groups.

9. The system of claim 8, wherein the multispectral LEDs are disposed on the illuminator in a spherical arrangement in which the sample is at the center.

10. The system of claim 8, wherein the multispectral LEDs comprise RGB LEDs.

11. The system of claim 10, wherein the multispectral LEDs further comprise other wavelengths in the visible spectrum as well as UV And IR.

12. The system of claim 7, wherein the optical transmission microscope further comprises a polarizing module, wherein the polarizing module comprises two polarizers which can be rotated independently.
